(19) United States
(12) Patent Application Publication
(10) Pub. No.: US 2026/0102320 A1
(43) Pub. Date: Apr. 16, 2026

(54) Agents for Restorative Treatment of Pulpitis and Management of Associated Pain, Kit Comprising the Same and Method of Use

(71) Applicant: Zachary PAN, Windsor (CA)

(72) Inventor: Zachary PAN, Windsor (CA)

(21) Appl. No.: 19/348,269

(22) Filed: Oct. 2, 2025

(30) Foreign Application Priority Data
Oct. 15, 2024 (US) 63707382

(51) Int. Cl. A61K 6/887; A61K 31/11; A61P 29/02; A61P 31/04

(52) U.S. Cl. CPC A61K 6/887; A61K 31/11; A61P 29/02; A61P 31/04

(57) ABSTRACT

The present disclosure provides a method of treating reversible and irreversible pulpitis and/or manage its associated pain in a primary or permanent tooth. The method comprises administering a therapeutically effective amount of a bioactive agent such as glutaraldehyde at an environment of a pH of about 7.0 to about 9.0 to an adult tooth followed by applying a therapeutically effective amount of a cavity liner or base onto the adult tooth that has been treated with the bioactive agent. A kit and a pharmaceutical composition for the treatment and associated pain management are also provided.

### CROSS REFERENCE TO RELATED APPLICATIONS

This application claims priority to U.S. Provisional Application No. 63/707,382 filed on Oct. 15, 2024, the entire contents of which is incorporated herein by reference.

### TECHNICAL FIELD

The present disclosure generally relates to the field of dentistry and, more particularly, to agents for restorative treatment of pulpitis, including symptomatic irreversible pulpitis and reversible pulpitis, and/or management of pain associated with pulpitis, a composition comprising the agents, a kit thereof, and a method of using the agents or composition for the restorative treatment and/or pain management of pulpitis.

### BACKGROUND

Dental caries remains the most prevalent disease among individuals of all ages. In its early stages, caries is typically asymptomatic, often unnoticed by patients or undetected during routine dental visits. Patients may become aware of caries due to sensitivity and pain as caries progresses to dentine and near the pulp, the innermost layer of a tooth, causing inflammation of the pulp, commonly known as pulpitis.

Pulpitis may be reversible or irreversible. Reversible pulpitis is usually a mild to moderate inflammation that can resolve with treatment. Patients can also manage pain associated with reversible pulpitis with over-the-counter analgesics, thus possibly allowing caries to further advance into the pulp chamber of a tooth to cause irreversible pulpitis.

Irreversible pulpitis may also be caused by tooth fracture, trauma and/or leaking restorations. Symptoms of irreversible pulpitis include spontaneous, constant and severe pain, which can significantly impact a patient's life. Consequently, many patients seek emergency dental treatment at this irreversible pulpitis stage.

The current preferred treatment option for irreversible pulpitis of a mature adult tooth is root canal treatment (“RCT”). RCT aims at alleviating severe pain and preserving the affected tooth by removing the whole contents of the pulp chamber and its root canal(s), and cleaning and sealing the root canal system. However, RCT is an intricate and time-consuming invasive procedure, and often accompanied by intra-operative and post-operative discomfort. In addition, its long-term success rate varies, influenced by multiple factors such as an operating dentist's skill in performing the RCT, inadequate care of the RCT treated tooth, or further trauma on the RCT treated tooth. Furthermore, a RCT treated teeth often requires a crown due to its increased susceptibility to fracture, requiring further visits and also complicating and escalating financial burdens for many patients.

In recent years, there has been a slight shift toward a more conservative vital pulp therapy (“VPT”), including pulpectomy and full pulpotomy. While a VPT has been reported to have a similar success rate as a RCT, the procedure is still an invasive endodontic procedure involving the removal of a tooth structure to gain access, and removal of partial or full pulp tissue, which weaken the tooth structure and increase fracture susceptibility.

Moreover, RCT, pulpectomy or full pulpotomy cannot preserve tooth vitality, which is critical in maintaining protective physiological responses to stimuli to a tooth. Both RCT and full pulpotomy relieve pain, but do not eliminate short-term post-op pain/tenderness or analgesic use in all cases. One primary reason for patients with pulpitis, especially symptomatic irreversible pulpitis, to seek dental treatment is to achieve immediate pain relief so that they can quickly resume their normal daily activities. However, prolonged pain may still persist in many patients after undergoing RCT and VPT. Moreover, the outcome of RCT and full pulpotomy depend on multiple operator- and case-level factors. Healing and success are sensitive to pre-op status, asepsis, anatomy of the tooth and may also be influenced by the skill of the operator. Finally, both procedures often require a crown to prevent fractures of the treated tooth, which is expensive.

Therefore, there is a need to provide a simple, conservative, and cost-effective alternative of treating pulpitis, especially irreversible pulpitis in an affected tooth, particularly an affected mature permanent tooth, and/or manage its associated pain, to overcome at least some of the deficiencies of the current treatment options.

### SUMMARY

In accordance with one aspect of the present disclosure, there is provided a kit for treating pulpitis and/or management of associated pain in a tooth. The tooth is a primary tooth or an adult (or permanent) tooth. The kit comprises a bioactive agent; a cavity liner/base, and instructions for using the kit.

In a further aspect, the bioactive agent is glutaraldehyde.

In a further aspect, the kit also comprises hydroxyethyl methacrylate (“HEMA”), which is provided with the bioactive agent in a pharmaceutical composition, contained in the cavity liner/base, or both. In some embodiments, the pharmaceutical composition comprises about 25% v/v to about 50% v/v of HEMA and about 2% v/v to about 10% v/v glutaraldehyde. In some embodiments, the glutaraldehyde is applied to the adult tooth at an environment of a pH of about 7 to about 9 to activate the glutaraldehyde. In some embodiments, a therapeutically effective amount of glutaraldehyde is about 5 μL to about 10 μL of 2% to 10% glutaraldehyde solution.

In a further aspect, the glutaraldehyde solidifies with the cavity liner/base to provide a semi-solid or solid bioactive glutaraldehyde carrier on a pulp floor or pulp chamber of the tooth.

In a further aspect, the cavity liner/base is a light curable liner/base. In some embodiments, the cavity liner/base is a commercially available liner/base for restorative dental treatments, including, but not limited to Vitrebond™, Virebond Plus™, or Activa™ BioACTIVE-RESTORATIVE™.

In still a further aspect, the pulpitis is reversible or irreversible pulpitis.

In accordance with another aspect of the present disclosure, there is provided a method of treating pulpitis and/or managing its associated pain in a tooth. The tooth is a primary tooth or an adult (permanent) tooth. The pulpitis is reversible or irreversible pulpitis. The method comprises administering a therapeutically effective amount of a bioactive agent at an environment of a pH of about 7.0 to about 9.0 to the adult tooth followed by applying a therapeutically effective amount of the cavity liner/base onto the adult tooth that has been treated with the bioactive agent. The pulpitis is reversible or irreversible pulpitis.

In a further aspect, the bioactive agent is glutaraldehyde. In some embodiments, a therapeutically effective amount of glutaraldehyde ranges from about 5 μL to about 10 μL of 2% to 10% glutaraldehyde solution. In some embodiments, the cavity liner/base is a light curable cavity liner or base. In some embodiments, the cavity liner/base is a commercially available liner/base for restorative dental treatments, including, but not limited to Vitrebond™, Virebond Plus™, or Activa™ BioACTIVE-RESTORATIVE™. In some embodiments, the cavity liner/base is mixed with the glutaraldehyde to provide a semi-solid or solid bioactive glutaraldehyde carrier.

In a further aspect, the method further comprises co-administering a therapeutically effective amount of HEMA with the therapeutically effective amount of the bioactive agent. In some embodiments, the bioactive agent is glutaraldehyde. In some embodiments, the glutaraldehyde and the HEMA are provided in a pharmaceutical composition, the concentration of the glutaraldehyde in the composition being about 5%-10% v/v and the concentration of the HEMA in the composition being about 25-50% v/v.

In accordance with yet another aspect of the present disclosure, there is provided a pharmaceutical composition for use to treat pulpitis and/or manage its associated pain in a tooth, comprising a bioactive agent and a cavity liner or base. In some embodiments, the bioactive agent is glutaraldehyde. In some embodiments, the cavity line or base is light curable.

### BRIEF DESCRIPTION OF THE DRAWINGS

Embodiments will now be described with reference to the appended figures:

FIG. 1A to 1J (collectively FIG. 1) show results of a pulpitis restorative protocol (“PRP”) treatment of irreversible pulpitis in accordance with an embodiment of the present disclosure. FIGS. 1A and 1B are bite wing (“BW”) radiograph and periapical (“PA”) radiograph showing tooth 26 of a patient upon diagnosis of irreversible pulpitis, respectively; and FIGS. 1C and 1D are BW radiograph and PA radiograph of the tooth 26 at 15 months post the PRP treatment, respectively. FIG. 1E is a PA radiograph taken after a second PRP treatment in accordance with an embodiment of the present disclosure, which was performed 4 days after the 15-month post-op when the patient complained about sensitivity to cold. FIG. 1F is a PA radiograph of the tooth at 18-month post the PRP treatment; FIGS. 1G and 1H are BW and PA radiograph of the tooth at 26 months post the first PRP treatment; and FIGS. 1I and 1J are BW and PA radiograph of the tooth at 39 months post the first PRP treatment.

FIG. 2A to 2F (collectively FIG. 2) show results of a PRP treatment of irreversible pulpitis in accordance with another embodiment of the present disclosure. FIG. 2A) and B) are BW and PA radiographs showing tooth 47 of a patient upon diagnosis of irreversible pulpitis; FIGS. 2C) and D) are BW and PA radiographs of the tooth 47 at 14 months post the PRP treatment; and FIGS. 2E) and F) are BW and PA radiographs of the tooth 47 at 30 months post the PRP treatment.

FIG. 3A to 3E (collectively FIG. 3) show results of a PRP treatment of reversible pulpitis in accordance with yet another embodiment of the present disclosure. FIGS. 3A and 3B are BW radiograph and PA radiograph showing tooth 46 of yet another patient upon diagnosis of reversible pulpitis, respectively; and FIGS. 3C and 3D are BW and PA radiographs of the tooth 46 at 25 months post PRP treatment, respectively; and FIG. 3E is a PA radiograph of the tooth 46 at 35 months post PRP treatment.

FIG. 4 shows results of an experiment testing the bactericide effect of glutaraldehyde.

### DETAILED DESCRIPTION

Unless defined otherwise, technical, scientific and medical terms used herein have the same meaning as commonly understood by one of ordinary skill in the art to which the present disclosure belongs.

As used herein, the term “treatment” refers to an approach for obtaining beneficial or desired clinical results. For the purposes described herein, beneficial or desired clinical results include, but are not limited to, alleviation of symptoms, diminishment of the extent of disease, stabilized (i.e., not worsening) state of disease such as pulpitis, delay or slowing of disease progression, amelioration or palliation of the disease state, and remission (whether partial or total), whether detectable or undetectable. Thus, “treatment” or “therapy” is an intervention performed to alter the pathology of a disorder or a disease state. Specifically, the treatment or therapy may directly prevent, slow down or otherwise decrease the progression of the diseases and render any symptoms associated with the disease such as pain to be manageable without further intervention for a prolonged period of time depending on what treatment chosen. For example, a cavity filling may last a few years while a crown or bridge may last a decade and an implant may last even longer.

The term “managing” refers to the process of controlling, monitoring, and treating the disease to improve a patient's quality of life, prevent complications, and slow the disease's progression.

The term “restorative treatment” means procedures that repair or restore a damaged, decayed, or missing tooth to restore its function, appearance, and/or structure.

As would be commonly understood by one of ordinary skill in the art to which the present disclosure belongs, when a tooth is “affected”, the tooth has been influenced or impacted by an external factor including, but not limited to, a condition, illness, or injury. When a tooth is “infected”, the infection is usually caused by a pathogen including virus, bacterium and fungus, leading to for example, and/or inflammation of the pulp tissue.

The terms “therapeutically effective amount”, “effective amount” or “sufficient amount” mean a quantity sufficient, when administered to a subject, including a mammal, for example, a human, to achieve the beneficial or desired result, for example, an amount effective to treat pulpitis in an affected/or infected adult tooth and/or manage pain associated with the pulpitis. Effective amounts of the agents described herein may vary according to factors such as the area of involved cavity of affected/infected tooth. Dosage or treatment regimens may be adjusted to provide the optimum therapeutic response, as is understood by a skilled person in the art to which this disclosure pertains.

Likewise, an “effective amount” of the agent or agents described herein refers to an amount sufficient to function as desired, such as to pulpitis in an affected/or infected adult tooth and/or manage pain associated with the pulpitis.

Administration “in combination with” one or more further therapeutic agents includes simultaneous (concurrent) and consecutive administration.

The term “pharmaceutically acceptable” means that the compound or combination of compounds is compatible with the remaining ingredients of a formulation for pharmaceutical use, including dental use, and that it is generally safe for administering to humans according to established governmental standards, including those promulgated by the United States Food and Drug Administration.

The term “carriers” as used herein include cosmetically or pharmaceutically acceptable carriers, excipients, or stabilizers that are non-toxic to the cell or subject being exposed thereto at the dosages and concentrations employed.

In understanding the scope of the present application, the articles “a”, “an”, “the”, and “said” preceding an element are intended to mean that there are one or more of the elements, and will be understood to mean “at least one” or “one or more”.

Additionally, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having”, and their derivatives.

In addition, all ranges given herein include the end of the ranges and also any intermediate-range points, whether explicitly stated or not.

Terms of degree such as “substantially”, “about” and “approximately” as used herein mean a reasonable amount of deviation of the modified term such that the end result is not significantly changed. For example, this term “about” can be construed as including a deviation of ±10 percent of the given numeric value provided such a deviation does not alter the end function or result of the value. Therefore, a value of about 1% can be construed to be a range from 0.9% to 1.1%. For the purposes of the present description and/or claims, and unless otherwise indicated, all numbers expressing quantities, percentages or proportions, and other numerical values used in the specification and claims, are to be understood as being modified in all instances by the term “about”.

The term “and/or” and the slash (/) punctuation mark can mean “and” or “or”.

At present, the preferred treatment of irreversible pulpitis in a mature adult tooth is RCT. However, this treatment requires removal of the contents of pulp chamber of the affected tooth and are invasive while the pain associated with pulpitis may still linger. In addition, a crown is often required to prevent the treated tooth from fracturing due to grinding and/or trauma.

To address at least some of these shortcomings, the present disclosure describes agents and method of using these agents for a restorative treatment of pulpitis and/or management of associated pain in a tooth and particularly, an adult tooth.

The agents and methods described herein can eliminate the need to open the pulp chamber of an affected or infected tooth to treat irreversible pulpitis. However, the agents and methods described herein can also be effective even if the pulp is exposed, for example, due to an accidental pulp exposure during the restoration.

In one aspect, the restorative method described herein comprises administering a therapeutically effective amount of a bioactive agent in an environment of a pH of about 7 to about 9 to an affected tooth, for example, a mature adult tooth, suffering from pulpitis followed by applying a therapeutically effective amount of a cavity liner or base onto the tooth that has been treated with the bioactive agent. As used herein, the term “bioactive agent” generally refers to a chemical substance that is bactericidal (i.e. killing bacteria). Examples of suitable bioactive agent include, but are not limited to, antiseptics, disinfectants and antibiotics.

In some embodiments, the bioactive agent is glutaraldehyde.

Glutaraldehyde (chemical structure shown below) is an organic compound and has two terminal aldehyde functional groups. Therefore, it can crosslink substances with primary amine groups, through condensation, via its aldehyde functional groups. As crosslinking can rigidify and deactivate proteins and other molecules that are critical for normal biological function, glutaraldehyde can induce apoptosis. In apoptosis, a cell breaks into several apoptotic bodies but the organelles are still functional. Hence, apoptosis can limit inflammation. The present disclosure hypothesizes that using glutaraldehyde to treat pulpitis can sterilize the infected dentine and adjacent pulp chamber, fix micro-abscessed pulp tissue adjacent to the caries, induce apoptosis, limit inflammation, and preserve the vitality of the affected tooth after treatment.

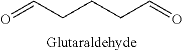

The crosslinking ability of glutaraldehyde can be affected by the pH environment and temperature. When in an acidic aqueous environment, glutaraldehyde is stable and none or weakly antimicrobial. Increasing the pH to a weak basic environment, it can significantly increase the compound's antimicrobial and fixative activity. At the same time, under a basic pH and body temperature (about 37° C.), glutaraldehyde quickly deactivates by self-polymerization and thus limits its crosslinking activity with further vital normal pulp tissue and make it safe in treatment of pulpitis. Therefore, it is desirable that the restorative treatment method as described herein utilizes a pH environment about 7 to about 9, for example, about 8.0 to about 8.5, so that glutaraldehyde can act on the affected or infected pulp tissues via its crosslinking to induce apoptosis while limiting its crosslinking activity with further vital normal pulp tissues, making it safe in treatment of pulpitis.

In one embodiment, a pH environment of about 8.2 is created for the application of glutaraldehyde by a repeated wash and treatment of the dental cavity with a solution of sodium hypochlorite (NaOCl, pH=about 8.2). The NaOCl solution can be prepared from a 1:1 volume to volume dilution of common household bleach containing 5 to 6% NaOCl such that the resulting solution with a pH about 11. Where necessary, a titration with an acid such as acetic acid can be employed to create a solution with the desirable pH of about 8.0 to about 8.5.

In some embodiments, a therapeutically effective amount of glutaraldehyde can be an amount that allows a sufficient coverage of the total dentine surface area of the cavity to be restored. The therapeutically effective amount of glutaraldehyde can be about 5 μL to about 10 μL, or about 10 μL, of 2% to 10% glutaraldehyde solution.

In some embodiments, glutaraldehyde can be applied as being present in a composition with 2-hydroxyethyl methacrylate (“HEMA”). For example, a composition sold under the trademark Gluma™, which contains about 5% to about 10% v/v glutaraldehyde and about 25% to about 50% v/v HEMA (pH=about 3 to about 4). In one embodiment, about 5 μL to about 10 μL of Gluma can be applied. In another embodiment, about 10 μL of Gluma can be applied.

In some embodiments, glutaraldehyde may be applied as a solution having a pH of about 7 to about 9. Such a solution may be prepared in situ by mixing the glutaraldehyde with an aqueous solution of pharmaceutically acceptable salt for a resulting solution having the desired basic pH. Where necessary, an acid such as acetic acid may be used for titration to create the desired basic pH. In some examples, 3% NaHCO₃ solution or NaOCl is used to adjust the acidic glutaraldehyde to the desired basic pH. Those skilled in the art would be familiar with choosing a suitable pharmaceutically acceptable salt. Examples of pharmaceutically acceptable salts include, but are not limited to, NaOCl or NaHCO₃. Such a solution may also be prepared in advance as a stable aqueous solution of glutaraldehyde having a pH of about 8.0 to about 8.5.

In another aspect, there is provided a method of treating pulpitis and/or manage its associated pain in a tooth. The tooth is a primary tooth or an adult tooth. The pulpitis is reversible or irreversible pulpitis. In some embodiments, the method comprises administering a therapeutically effective amount of a bioactive agent, such as glutaraldehyde, at an environment of a pH of about 7.0 to about 9.0 to the tooth followed by applying a therapeutically effective amount of a cavity liner/base. In an example, a therapeutically effective amount of glutaraldehyde ranges from about 5 μL to about 10 μL of 2% to 10% glutaraldehyde solution, a layer of a cavity base/liner is then applied on top of the glutaraldehyde so that both layers are mixed and solidified by a curing light. Upon curing, a solid glutaraldehyde containing bioactive carrier forms. In some embodiments, the cavity liner/base is Vitrebond™ or Virebond Plus™.

According to the present disclosure, the treatment method further comprises overlaying the glutaraldehyde applied to the treated tooth with a cavity liner/base. A light curable cavity liner/base can be applied. For example, Vitrebond™ powder and liquid comprising a light cure resin modified glass ionomer liner/base and HEMA can be used. In another embodiment, Vitrebond™ Plus, having a similar composition as Vitrebond™ powder and liquid, can be used.

Without being limited to any particular theory, a hypothetical mechanism underlying the treatment method described herein involves: 1) once activated under a basic condition, glutaraldehyde sterilizing infected dentine and eradicating microorganisms; 2) glutaraldehyde inducing apoptosis and fixing micro-necrotic pulp tissue near the caries; 3) glutaraldehyde and its combination use with a cavity liner/base such as Vitrebond™ solidifying the bioactive agent such as glutaraldehyde. It is believed that the solidified glutaraldehyde carrier acts as a bioactive agent, which limits the self-polymerization of glutaraldehyde monomer and allows the more potent glutaraldehyde monomer vapor to leach out the carrier to crosslink inner amino acids of the proteins, thus increasing the local concentration of glutaraldehyde, providing different chemical activity and/or helping to seal the dental cavity caused by caries to prevent subsequent bacterial invasion.

It is further hypothesized that the mechanism of the instant pain relief with the treatment method described herein appears to include: 1) apoptosis to avoid further inflammation as discussed above; and 2) total extermination of invading bacteria and deactivation of the bacterial toxin by the protein cross-linking activity of glutaraldehyde.

Additionally, in some embodiments, glutaraldehyde's occlusive action on dentinal tubules due to its cross-linking with the proteins inside the tubules, also in conjunction with HEMA and a cavity liner/base such as Vitrebond™, aids in sealing these structures and may help in reducing the pain.

Further, in some embodiments, some glutaraldehyde may be mixed with a cavity liner/base such as Vitrebond so that glutaraldehyde can be solidified by light cure after covered with the cavity liner/base.

In one embodiment, the bioactive agent such as glutaraldehyde and the cavity liner/base may be present in a kit for a restorative treatment of pulpitis and/or management of associated pain in a primary or permanent tooth. The kit may be supplied with instructions for using the kit.

In another embodiment, there is provided a composition for treating pulpitis and/or management of associated pain, the comprising a therapeutically effective amount of a bioactive agent and a cavity liner/base. In some examples, the bioactive agent is glutaraldehyde. In some embodiments, the cavity line/base is a light curable cavity liner/base.

In another embodiment, a therapeutically effective amount of a bioactive agent is less than about 10 microliter (μL), for about 5 μL to about 10 μL. A therapeutically effect amount of the cavity liner/base is about 0.5 to about 1.0 mm in thickness.

In another embodiment, the restorative pulpitis treatment method may be applied to an affected primary tooth suffering from either reversible or irreversible pulpitis.

When compared with VPT and RCT, the restorative pulpitis treatment method described herein can possess at least one or more of the following advantages:
possibility to preserve tooth vitality, as no contents may be removed from an affected tooth's pulp chamber;
immediate pain relief following the treatment without taking any pain killers or analgesics;
ability to preserve tooth structure without need to remove the tooth structure to access pulp, and render tooth more longevity and better strength;
quick recovery time;
cost effectiveness as RCT and pulpectomy or pulpotomy are expensive and constitute an extra dental procedure in addition to the restoration;
easy to perform and ability to be performed by general dentists, resulting in less waiting time for patients and less chair time for dentists; and
no need for a dentist to make a differential diagnosis between reversible and irreversible pulpitis, as the treatment method can be used to treat both conditions.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

This example describes a general PRP to treat pulpitis and/or manage its associated pain. The tooth numbering in this Example is according to FDI World Dental Federation notation.

patients were treated: 17 male and 10 female, age of 15 to 71 with average age of 39 years old. 16 had IP and 11 cases had reversible pulpitis (RP) were treated with PRP at a dental clinic from March 2022 to April 2025. This report includes cases with varied follow-up durations: 6 cases with follow-ups up from 25-36 months, 14 cases between 13 to 24 months, and 4 cases from 7-12 months and 3 cases from 3-6 months at the time of drafting up this case series report. The average follow-up duration was 19 months.

The PRP generally followed the following procedure:
a. prepare the dental cavity by removing carious enamel and peripheral dentine to an adequate restorative cavity form with a high-speed diamond bur under water spray cooling as conventional restoration after local anaesthesia. Where necessary, prior to the restoration, the dental cavity may be shaped and cleaned;
b. continue to remove carious tissue on cavity peripheral walls with a slow speed round caribe bur, then with a hand spoon excavator to hard enamel, hard dentine or cementum so that about 2 mm good bonding peripheral seal zone for the restoration is available;
c. remove carious tissue overlaying the pulp chamber and pulp horns of teeth very carefully and selectively when the dentine thickness between cavity and pulp is thinner than about 1.0 mm on intro-oral radiographs, no round bur should be used over pulp roof and only hand spoon excavator is used. Some soft dentine can be left in situ to avoid any perforation or pulp exposure when the dentine is very thin between caries and pulp;
d. before, during, and after the carious removal procedure, rinse the cavity frequently with 2.5% (w/v) concentration of sodium hypochlorite (NaOCl), pH about 8.2, derived from a 1:1 dilution of about 5.25% household bleach with water;
e. after completion of the cavity preparation, place a matrix band, pack the cavity gently with a cotton pellet socked with NaOCl pH about 8.2 and leave the cotton pellet for about 3 minutes to create a local pH of about 7 to about 9, preferably between about 7.5 to about 8.5, and rinse the cavity with water after the cotton pellet is removed;
f. etch the cavity with 37% phosphoric acid, about 30 seconds on enamel and about 15 seconds on dentine and cementum, rinse the cavity for about 10 seconds with distilled water, air dry it with air syringe, rinse the cavity again with about 2.5% (w/v) concentration of NaOCl), pH about 8.2, and dry the cavity briefly again with air syringe (if Amalgam restoration is used, this step f acid etching can be omitted);
g. apply a coat (about 5 to about 10 μL depending on the total dentine surface area of the cavity to be restored) of a PRP agent which contains about 5%-10% v/v glutaraldehyde (“GA”) and optionally, about 25%-50% v/v 2-hydroxyethyl methacrylate (“HEMA”) to cover the dentine surface of the cavity with a micro brush applicator saturated with GA, leave it for about a minute, and during the waiting time, mix Vitrebond (3M, U.S.A.) powder and liquid at 1:1 ratio, or mix Vitrebond Plus Clicker with equal amount from each tube, apply a thin layer (about 0.5 mm-1.0 mm thickness) of Vitrebond with a Dycal™ applicator over the PRP agent on the dentine surface overlaying pulp chamber and pulp horns, and light cure Vitrebond for about 20 seconds with a 1200 W/cm² intensity LED light;
h. optionally, apply a thin layer of dental adhesive material (3M Single Bond or Universal Adhesive, 3MESPE St Paul, USA) with a new micro brush applicator to the entire cavity wall surfaces including Vitrebond surface and enamel, rubbing for few seconds, and light cure the adhesive for 5 seconds with a 1200 W/cm² intensity LED light;
i. optionally, place a thin layer of flowable composite resin in needle syringe to cover the cavity floor including Vitrebond, and cure the flowable composite resin for about 20 seconds with a 1200 W/cm² intensity LED light;
j. optionally, restore the remaining cavity with a composite resin in small incremental and light cure for about 20 second each incremental with a 1200 W/cm² intensity LED light or restored the cavity with Amalgam; and
k. optionally, adjust occlusion and remove any high spots and polished with polishing disc.

Results: treatment success was assessed based on the absence of pain, absence of soft tissue abnormality, positive responses to cold vitality tests and lack of apical lesion in radiographic images months following the PRP. In all cases treated with the PRP, there seems to be no tertiary dentin formation found on radiographs. This finding may also support that the PRP treatment suppresses pulp inflammation.

10 of the 11 cases with RP and 14 of the 16 cases with IP treated with PRP according to the embodiments were symptom free and retained pulp vitality at their final assessment. 88.9% treatment success across all cases was observed. Notably, 74% of the cases reported minimal or no pain (0/10 to 1/10) in the same evening post-PRP treatment, and 96% of the cases reported no pain 24 hours post-PRP treatment without analgesics. All the 24 successful cases had positive response to the cold vitality test at their final visit. 23 cases showed no change in PA radiographs.

One case failed at 24-months after the PRP treatment likely due to failed restoration following sensitivity to cold at 18-months. There was leakage at interproximal margin which was placed below Cement-Enamel Junction (CEJ) and visible on the radiograph. Recommendation of re-do the restoration was declined by the patient and tooth failed at 24 months. The causes of the other two cases failed at sixteen-months and thirty-one months respectively were not clear, but likely also due to fracture of the restoration.

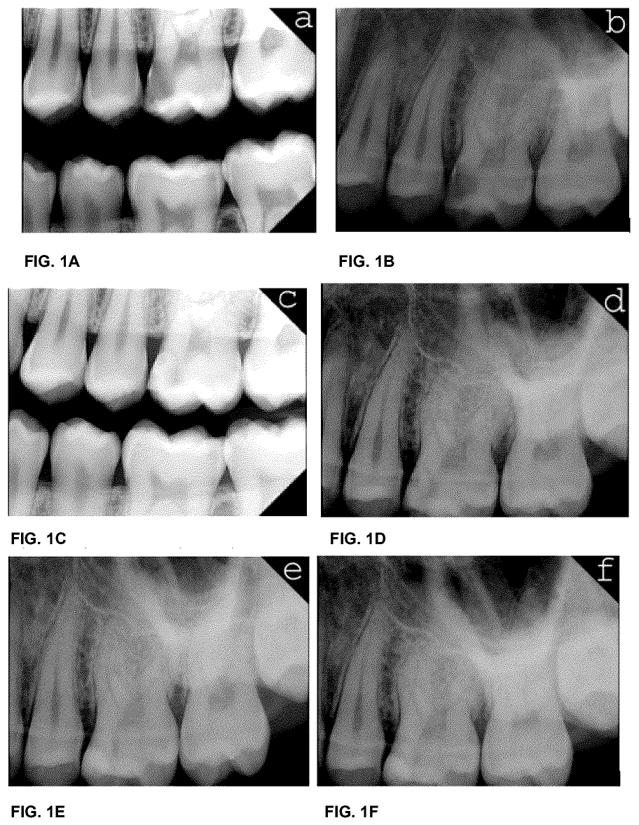

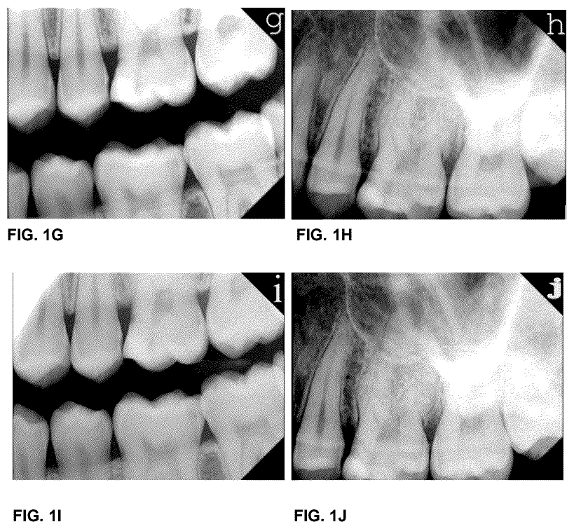

In one case, a 16-year-old male with tooth 26 deep caries with symptom of irreversible pulpitis, was treated successfully (see FIGS. 1A to 1J). Both the BW and PA radiographs showed that tooth 26 dental caries was large and deep touching mesial pulp horn (see FIGS. 1A and 1B). However, as shown in FIGS. 1G and 1H, at 26 months post the PRP treatment, and FIGS. 11 and 1J at 39-month post the PRP treatment, although the lamina dura appeared “ballooned” at the apex possibly linked to twice PRP treatments (see FIGS. 1C to 1E), there was no typical radiolucency suggestive of a periapical abscess, and the tooth remained asymptomatic and vital, responding normally to cold vitality testing.

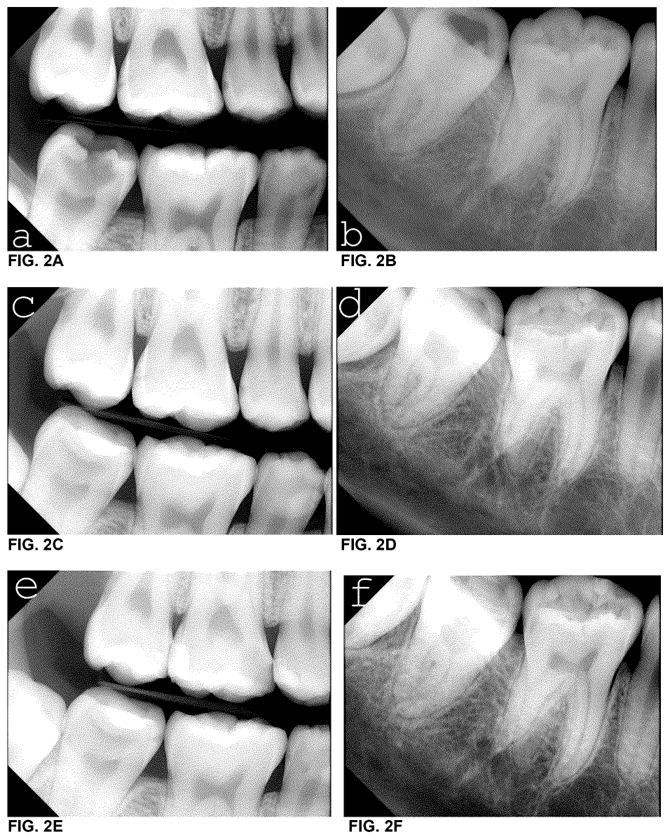

In one case, a 15-year-old female with tooth 47 deep caries with symptom of irreversible pulpitis, was treated successfully (see FIGS. 2A to 2F). As shown in FIGS. 2C to 2F, no visible apical pathology was observed at 18 and 30 months post the PRP treatment.

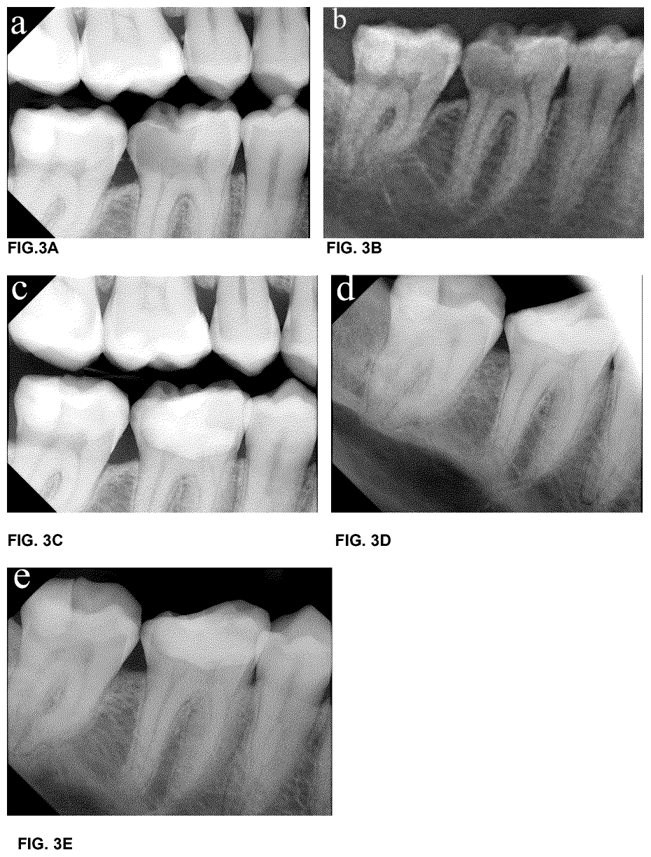

In one case, a 33-year-old male with tooth 46 gross caries with symptom of reversible pulpitis was treated successfully (see FIGS. 3A to 3E). Both the BW and PA radiographs showed that tooth 46 dental caries was large and deep in the proximity of pulp. As shown in FIGS. 3C to 3E, at 25-month and 35-month post the PRP treatment, there was no apical pathology visible, no pathologic sign developed around tooth 46 apexes and no dentine bridge formation was visible in tooth 46 pulp chamber, and the tooth remained vital.

The results of the treatment using embodiments of the methods described herein are shown in Table and Table 2 below.

TABLE 1
Results of PRP Treatment

| | | | | Pre- | | | Pre-Op | | | | | | | |
|---|---|---|---|---|---|---|---|---|---|---|---|---|---|---|
| Case | tooth | | | Op | | | Cold | Spontaneous | | Post-Op | Post-Op | Post-Op | Cold | Duration |
| # | # | Sex | age | Pain | Sponteno | STP | Test | Pain | | Pain | Symptom | Radiograph | Test | Observation |
| 1 | 26 | M | 17 | 6-7 | Yes | Yes | Painful | <12 | hrs | 0 | No pain, no | Atypica Apex | Normal | 25-36 |
| | | | | | | | | | | | STP | change | | Months |
| 2 | 47 | F | 15 | 7 | Yes | Yes | Painful | Few | hrs | 0 | No sign & | No change at | Normal | 25-36 |
| | | | | | | | | | | | symptoms | Apex | | Months |
| 3 | 46 | M | 33 | 0-1 | No | No | Sensitive | NA | | 0 | No sign & | No change at | Normal | 25-36 |
| | | | | | | | | | | | symptoms | Apex | | Months |
| 4 | 16 | F | 39 | 2-3 | No | No | Sensitive | NA | | 0 | No sign & | Atypica Apex | Normal | 25-36 |
| | | | | | | | | | | | symptoms | change | | Months |
| 5 | 26 | M | 41 | 0-1 | No | No | Senstive | NA | | 0 | Pain and | No change at | Negative | 25-36 |
| | | | | | | | | | | | STP, | Apex | | Months |
| 6 | 15 | F | 38 | 8 | Yes | Yes | Painful | 1 | hour | 0 | Pain and | Apical | Negative | 13-24 |
| | | | | | | | | | | | STP, | Lucency | | Months |
| 7 | 14 | M | 32 | 8 | Yes | Yes | Painful | 12-24 | hrs | 0 | No sign & | No change at | Normal | 25-36 |
| | | | | | | | | | | | symptoms | Apex | | Months |
| 8 | 26 | M | 30 | 7 | Yes | Yes | Painful | 2 | hours | 0 | Pain and | No change at | Negative | 13-24 |
| | | | | | | | | | | | STP, | Apex | | Months |
| 9 | 17 | M | 48 | 6-7 | Yes | Yes | Painful | <12 | hrs | 0 | No sign & | No change at | Normal | 13-24 |
| | | | | | | | | | | | symptoms | Apex | | Months |
| 10 | 14 | F | 24 | 2 | No | No | Sensitive | NA | | 0 | No sign & | No change at | Normal | 13-24 |
| | | | | | | | | | | | symptoms | Apex | | Months |
| 11 | 24 | M | 40 | 7 | Yes | Yes | Painful | <24 | hrs | 2 | No sign & | No change at | Normal | 13-24 |
| | | | | | | | | | | | symptoms | Apex | | Months |
| 12 | 38 | F | 54 | 0 | No | No | Sensitive | NA | | 0 | No sign & | No change at | Normal | 13-24 |
| | | | | | | | | | | | symptoms | Apex | | Months |
| 13 | 15 | M | 22 | 8 | Yes | Yes | Painful | 24-36 | hrs | 0 | Cold senstive, | No change at | senstive | 13-24 |
| | | | | | | | | | | | No pain | Apex | | Months |
| 14 | 27 | M | 67 | 2 | No | No | Sensitive | NA | | 0 | No sign & | No change at | Normal | 13-24 |
| | | | | | | | | | | | symptoms | Apex | | Months |
| 15 | 17 | F | 57 | 8-9 | Yes | STP | Lingering | 24 | hrs | 0 | No sign & | No change at | Normal | 13-24 |
| | | | | | | | pain | | | | symptoms | Apex | | Months |
| 16 | 15 | M | 34 | 9 | Yes | Yes | Pain | 24 | hrs | 1 | Cold senstive, | No change at | senstive | 13-24 |
| | | | | | | | | | | | No pain | Apex | | Months |
| 17 | 26 | F | 40 | 2 | No | No | Sensitive | NA | | 0 | No sign & | No change at | Normal | 13-24 |
| | | | | | | | | | | | symptoms | Apex | | Months |
| 18 | 26 | M | 40 | 4 | No | yes | Lingering | NA | | 0 | Cold senstive, | No change at | senstive | 13-24 |
| | | | | | | | pain | | | | No pain | Apex | | Months |
| 19 | 28 | F | 59 | 6 | Yes | Yes | Very | Few | hrs | 0 | No sign & | No change at | Normal | 13-24 |
| | | | | | | | Painful | | | | symptoms | Apex | | Months |
| 20 | 15 | M | 23 | 5 | No | Yes | Very | NA | | 0 | No sign & | No change at | Normal | 13-24 |
| | | | | | | | Senstive | | | | symptoms | Apex | | Months |
| 21 | 25 | F | 35 | 7 | Yes | Yes | Painful | <24 | hrs | 0 | No sign & | No change at | Normal | 7-12 |
| | | | | | | | | | | | symptoms | Apex | | Months |
| 22 | 48 | M | 48 | 2 | No | No | Painful | NA | | 0 | Cold senstive, | No change at | senstive | 7-12 |
| | | | | | | | | | | | No pain | Apex | | Months |
| 23 | 37 | M | 71 | 6 | No | No | Normal | NA | | 0 | No sign & | No change at | Normal | 7-12 |
| | | | | | | | | | | | symptoms | Apex | | Months |
| 24 | 34 | M | 47 | 7-8 | Yes | Yes | Painful | 24 | hrs | 0 | No sign & | Disappearing | Normal | 7-12 |
| | | | | | | | | | | | symptoms | PDL | | Months |
| 25 | 46 | M | 17 | 8 | Yes | Yes | Sensitive | 24 | hrs | 0 | No sign & | No change at | Normal | 3-6 |
| | | | | | | | | | | | symptoms | Apex | | Months |
| 26 | 27 | M | 34 | 7-8 | Yes | Yes | Painful | 48 | hrs | 0 | No sign & | No change at | Normal | 3-6 |
| | | | | | | | | | | | symptoms | Apex | | Months |

| | | | | | | | | | | | | | |
|---|---|---|---|---|---|---|---|---|---|---|---|---|---|
| 27 | 17 | F | 52 | 8 | yes | Yes | Painful | < or =24 | 0 | Sensitive | No change at | senstive | 3-6 |
| | | | | | | | | hrs | | to cold | Apex | | Months |

TABLE 2
Pain Reduction after PRP Treatment

| | | | | Pre-Operative | | | | Post-Operative | | |
|---|---|---|---|---|---|---|---|---|---|---|
| Patient Information | | | | VAS Pain | Spontaneous | Spontaneous | | Same | 24 | 48 |
| Case # | Tooth# | Sex | Age | Severity 0-10 | Pain | Pain duration | | night | Hr | Hr |
| 1 | 26 | M | 17 | 6-7 | Yes | <12 | hrs | 0 | 0 | 0 |
| 2 | 47 | F | 15 | 7 | Yes | Few | hrs | 0 | 0 | 0 |
| 3 | 46 | M | 33 | 0-1 | No | NA | | 0 | 0 | 0 |
| 4 | 16 | F | 39 | 2-3 | No | NA | | 0 | 0 | 0 |
| 5 | 26 | M | 41 | 0-1 | No | NA | | 0 | 0 | 0 |
| 6 | 15 | F | 38 | 8 | Yes | 1 | hour | 0 | 0 | 0 |
| 7 | 14 | M | 32 | 8 | Yes | 12-24 | hrs | 0 | 0 | 0 |
| 8 | 26 | M | 30 | 7 | Yes | 2 | hours | 0 | 0 | 0 |
| 9 | 17 | N | 48 | 6-7 | Yes | <12 | hrs | 0 | 0 | 0 |
| 10 | 14 | F | 24 | 2 | No | NA | | 0 | 0 | 0 |
| 11 | 24 | N | 40 | 7 | Yes | <24 | hrs | 4 | 2 | 0 |
| 12 | 38 | F | 54 | 0 | No | NA | | 0 | 0 | 0 |
| 13 | 15 | M | 22 | 8 | Yes | 24-36 | hrs | 1 | 0 | 0 |
| 14 | 27 | M | 67 | 2 | No | NA | | 1 | 0 | 0 |
| 15 | 17 | F | 57 | 8-9 | Yes | 24 | hrs | 0 | 0 | 0 |
| 16 | 15 | M | 34 | 9 | Yes | 24 | hrs | 5 | 1 | 0 |
| 17 | 26 | F | 40 | 2 | No | NA | | 0 | 0 | 0 |
| 18 | 26 | M | 40 | 4 | No | NA | | 0 | 0 | 0 |
| 19 | 28 | F | 59 | 6 | Yes | Few | hrs | 4 | 0 | 0 |
| 20 | 15 | M | 23 | 5 | No | NA | | 0 | 0 | 0 |
| 21 | 25 | F | 35 | 7 | Yes | <24 | hrs | 0 | 0 | 0 |
| 22 | 48 | M | 48 | 2 | No | NA | | 0 | 0 | 0 |
| 23 | 37 | M | 71 | 6 | No | NA | | 2 | 0 | 0 |
| 24 | 34 | M | 47 | 7-8 | Yes | 24 | hrs | 4 | 0 | 0 |
| 25 | 46 | M | 17 | 8 | Yes | 24 | hrs | 4 | 0 | 0 |
| 26 | 27 | M | 34 | 7-8 | Yes | 48 | hrs | 0 | 0 | 0 |
| 27 | 17 | F | 52 | 8 | Yes | < or =24 | hrs | 2 | 0 | 0 |

The PRP described herein may be indicated and contra-indicated as shown in Table 3 below:

TABLE 3
Indications and Contra-indications for PRP

| Indications | Contra-indications |
|---|---|
| Deep caries near or touch pulp on radiographs | Necrotic pulp (negative response to pulp |
| | test) |
| Symptomatic irreversible pulpitis | Pulp exposure with uncontrollable pulp |
| | bleeding |
| Symptomatic reversible pulpits | History of repeated spontaneous and |
| | severe pain of the same tooth |
| Positive response to pulp vitality test. | Spontaneous and constant, severe pain |
| | lasting longer than 24 hours |
| Normal periapical tissue radiographically | Severe Sensitive to Percussion (STP) |
| | associated with palpation pain at the root |
| | apex |
| Normal buccal or lingual soft tissue, no swollen, | Presence of soft tissue swelling or sinus |
| lumps or fistula | tract/fistula |
| Restorable tooth structure | Radiographic evidence of periapical |
| | pathology |
| Patient preference for conservative treatment | Immunocompromised patients (relative) |

### Example 2

This example describes the bactericide effect of bioactive glutaraldehyde carrier.

Materials and procedure: 1) sterile Petri dishes 60 mm; 2) sterile mini glass dishes 12 mm; 3) sterile absorbative paper circle 8 mm; 4) Tryptic Soy Agar (Innovating Science); 5) oral bacteria; 6) GA+HEMA pH8.0; 7) Vitrebond Plus (3M).

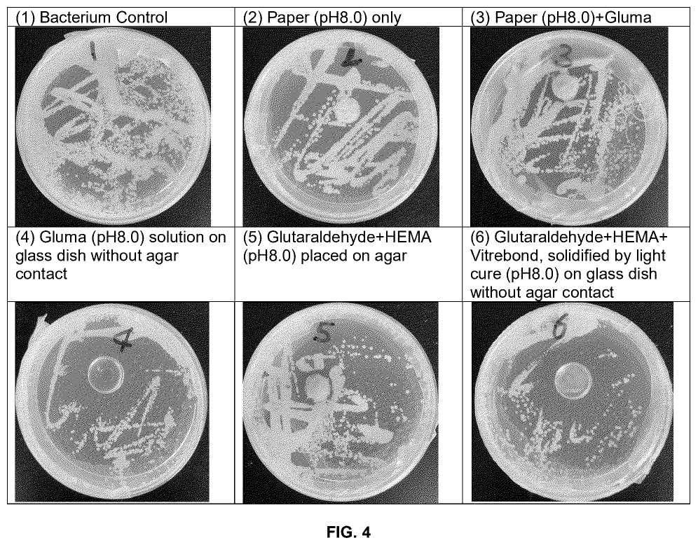

The procedure was as follows: inoculate oral bacteria to Tryptic Soy agar Petric dishes with a metal inoculating loop (FIG. 4(1): control); place a sterile absorbative paper circle loaded with 10 ul of distill water (pH8.0) to Petri dish #2 (FIG. 4(2)); add 10 μl Gluma (pH8.0) to Petri dish #3 (FIG. 4(3)) and GA-HEMA (pH8.0) to Petri dish #5 (FIG. 4(5)); adhere the sterile absorbative paper circle directly to the agar surface; turn all the Petri dishes #1, 2, 3 and 5 with agar on top; load a small glass dish with 10 ul GA+HEMA, pH8.0 in Petri dish #4 (FIG. 4(4)); and another small glass dish with 10 μl GA+HEMA, pH8.0, then Vitrebond Plus over the GA+HEMA on the glass dish followed by light cure in Petri dish #6 (FIG. 4(6)). In Petri dishes #4 and 6, the glass dishes stayed on bottom facing agar. All the petri dishes were air tight sealed with Parafilm and incubated at 37 degree for 24 hours.

As shown in FIG. 4, glutaraldehyde showed bactericide effect as seen in the bacterial agar culture on Petri dishes #3, 4, 5 and 6. However, direct contact with agar had less bactericide effect (very small inhibition rings around paper loaded with glutaraldehyde) on Petri dishes #3 and 5. The very weak bactericide effect of direct contact of GA with agar may result from the reaction of glutaraldehyde with amino acids of the protein in Tryptic Soy agar and depleted its functional groups. The glutaraldehyde on glass dishes which had no direct contact with the agar and bacteria (see Petri dishes #4 and 6), however, showed strong bactericide effect, as a large no-bacteria zone was observed above the dishes and significant less bacterium growth was observed in the entire dishes.

The results showed that the glutaraldehyde monomers from both the glutaraldehyde solution and solid GA can react with the amino acids of the proteins of the bacteria inoculated on the top agar plate to deactivate the bacteria.

The results also showed that the solid glutaraldehyde carrier has a potent biocide effect and is an effective agent in the PRP treatment of IP and RP. It is believed that a solid glutaraldehyde carrier has the following advantages: 1) preventing GA polymerization; and 2) defusing or releasing more potent monomers to eliminate bacteria nearby even without direct contact with the bacteria. A solid glutaraldehyde carrier may be more potent for the PRP treatment that a liquid glutaraldehyde.

Numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will be appreciated that the examples used herein are for illustrative purposes only. Different configurations and terminology can be used without departing from the principles expressed herein.

Although the above principles have been described with reference to certain specific examples, various modifications thereof will be apparent to those skilled in the art as having regard to the appended claims in view of the specification as a whole.

All publications, patents, and patent applications cited herein are incorporated by reference in their entirety to the same extent as if each publication, patent, or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

### REFERENCES

American Association of Endodontists (2013) ‘Guide to Clinical Endodontics’, American Association of Endodontists.
Asgary, S. et al. (2013) ‘One-year results of vital pulp therapy in permanent molars with irreversible pulpitis: an ongoing multicenter, randomized, non-inferiority clinical trial’, Clinical Oral Investigations, 17(2), pp. 431-439.
Asgary, S. et al. (2015) ‘Five-year results of vital pulp therapy in permanent molars with irreversible pulpitis: a non-inferiority multicenter randomized clinical trial’, Clinical Oral Investigations, 19(2), pp. 335-341.
Chiu, I. M. et al. (2013) ‘Bacteria activate sensory neurons that modulate pain and inflammation’, Nature, 501(7465), pp. 52-57.
Davis, M. J., Myers, R. and Switkes, M. D. (1982) ‘Glutaraldehyde: an alternative to formocresol for vital pulp therapy’, ASDC journal of dentistry for children, 49(3), pp. 176-180.
Dugas, N. N. et al. (2003) ‘Periapical health and treatment quality assessment of root-filled teeth in two Canadian populations’, International Endodontic Journal, 36(3), pp. 181-192.
Edwards, D. et al. (2024) ‘The use of medicaments in the management of symptomatic irreversible pulpitis: A community-based cohort study’, International Endodontic Journal, 57(4), pp. 416-430.
Falatah, A. M. et al. (2023) ‘Comprehensive Strategies in Endodontic Pain Management: An Integrative Narrative Review’, Cureus, 15(12), p. e50371
Gough, J. E., Scotchford, C. A. and Downes, S. (2002) ‘Cytotoxicity of glutaraldehyde crosslinked collagen/poly(vinyl alcohol) films is by the mechanism of apoptosis’, Journal of Biomedical Materials Research, 61(1), pp. 121-130.
Hojo, S. et al. (1994) ‘Acid profiles and pH of carious dentin in active and arrested lesions’, Journal of Dental Research, 73(12), pp. 1853-1857.
Huang, D. et al. (2024) ‘Expert consensus on difficulty assessment of endodontic therapy’, International Journal of Oral Science, 16(1), p. 22.
Kazeminia, M. et al. (2020) ‘Dental caries in primary and permanent teeth in children's worldwide, 1995 to 2019: a systematic review and meta-analysis’, Head & Face Medicine, 16(1), p. 22.
Kulzer, 2023 gluma-desensitizer-faqs--h2-wp-en---mdr-website.pdf′ (no date). Available at: https://www.kulzer.com/media/product-downloads/international-en-int2/gluma/gluma-desensitizer/gluma-desensitizer-faqs--h2-wp-en---mdr-website.pdf.
Migneault, I. et al. (2004) ‘Glutaraldehyde: behavior in aqueous solution, reaction with proteins, and application to enzyme crosslinking’, BioTechniques, 37(5), pp. 790-796, 798-802.
Ng, Y.-L. et al. (2004) ‘Prevalence of and factors affecting post-obturation pain in patients undergoing root canal treatment’, International Endodontic Journal, 37(6), pp. 381-391.
Ng, Y.-L., Mann, V. and Gulabivala, K. (2010) ‘Tooth survival following non-surgical root canal treatment: a systematic review of the literature’, International Endodontic Journal, 43(3), pp. 171-189.
Ricucci, D., Loghin, S. and Siqueira, J. F. (2014) ‘Correlation between Clinical and Histologic Pulp Diagnoses’, Journal of Endodontics, 40(12), pp. 1932-1939.
Salem, M., Mauguen, Y. and Prange, T. (2010) ‘Revisiting glutaraldehyde cross-linking: the case of the Arg-Lys intermolecular doublet’, Acta Crystallographica. Section F, Structural Biology and Crystallization Communications, 66 (Pt 3), pp. 225-228.
Scaffidi, P., Misteli, T. and Bianchi, M. E. (2002) ‘Release of chromatin protein HMGB1 by necrotic cells triggers inflammation’, Nature, 418(6894), pp. 191-195.
Scheffel, D. L. S. et al. (2015) ‘Transdentinal cytotoxicity of glutaraldehyde on odontoblast-like cells’, Journal of Dentistry, 43(8), pp. 997-1006.
Sehmi, S. K. et al. (2016) ‘The bactericidal activity of glutaraldehyde-impregnated polyurethane’, MicrobiologyOpen, 5(5), pp. 891-897.
Signor, B. et al. (2024) ‘Postoperative pain after single-visit root canal treatment or vital pulp therapy: A systematic review and meta-analysis’, Journal of the American Dental Association (1939), 155(2), pp. 118-137.e1.
Sjôgren, U. et al. (1997) ‘Influence of infection at the time of root filling on the outcome of endodontic treatment of teeth with apical periodontitis’, International Endodontic Journal, 30(5), pp. 297-306.
Thomas, S. and Russell, A. D. (1974) ‘Temperature-induced changes in the sporicidal activity and chemical properties of glutaraldehyde’, Applied Microbiology, 28(3), pp. 331-335.
Udoye, C. I., Sede, M. A. and Jafarzadeh, H. (2014) ‘The pattern of fracture of endodontically treated teeth’, Trauma Monthly, 19(4), p. e17054.
Watt, R. G. et al. (2019) ‘Ending the neglect of global oral health: time for radical action’, The Lancet, 394(10194), pp. 261-272.
Yang, L. et al. (2015) ‘KIM-1-mediated phagocytosis reduces acute injury to the kidney’, The Journal of Clinical Investigation, 125(4), pp. 1620-1636.
Zenaldeen, R. et al. (2023) ‘Partial Pulpotomy in Mature Permanent Molars with Symptoms Indicated Irreversible Pulpitis Using MTA: A Study of Three Case Reports over Four-Year Follow-Up’, Case Reports in Dentistry, 2023, p. 1344101.

1. A kit for treating pulpitis and/or management of associated pain in a tooth, comprising:
a bioactive agent;
a cavity liner or base; and
instructions for using the kit.

2. The kit of claim 1, wherein the bioactive agent is glutaraldehyde.

3. The kit of claim 1, further comprising hydroxyethyl methacrylate (“HEMA”), the HEMA being provided with the bioactive agent in a pharmaceutical composition, contained in the cavity liner or base, or both.

4. The kit of claim 2, wherein the glutaraldehyde solidifies with the cavity liner or base to provide a semi-solid or solid bioactive glutaraldehyde carrier on a pulp floor or pulp chamber of the tooth.

5. The kit of claim 2, wherein the glutaraldehyde is applied to the tooth at an environment of a pH of about 7 to about 9 to activate the glutaraldehyde.

6. The kit of claim 2, wherein a therapeutically effective amount of glutaraldehyde is about 5 μL to about 10 μL of 2% to 10% glutaraldehyde solution.

7. The kit of claim 1, wherein the cavity liner or base is a light curable liner.

8. The kit of claim 3, wherein the pharmaceutical composition comprises about 25% v/v to about 50% v/v of HEMA and about 2% v/v to about 10% v/v the bioactive agent, the bioactive agent being glutaraldehyde.

9. The kit of claim 1, wherein the pulpitis is reversible or irreversible pulpitis.

10. The kit of claim 1, wherein the tooth is a primary or permanent tooth.

11. A method of treating pulpitis and/or manage its associated pain in a tooth, the method comprising administering a therapeutically effective amount of a bioactive agent at an environment of a pH of about 7.0 to about 9.0 to the tooth followed by applying a therapeutically effective amount of a cavity liner or liner onto the tooth that has been treated with the bioactive agent.

12. The method of claim 11, wherein the bioactive agent is glutaraldehyde.

13. The method of claim 11, wherein the method further comprising co-administering a therapeutically effective amount of HEMA with the therapeutically effective amount of the bioactive agent.

14. The method of claim 12, wherein the therapeutically effective amount of glutaraldehyde ranges from about 5 μL to about 10 μL of 2% to 10% glutaraldehyde solution.

15. The method of claim 13, wherein the bioactive agent and the HEMA are provided in a pharmaceutical composition, the bioactive agent is glutaraldehyde, the concentration of the glutaraldehyde in the composition being about 5%-10% v/v and the concentration of the HEMA in the composition being about 25-50% v/v.

16. The method of claim 11, wherein the cavity liner or base is a light curable cavity liner or base.

17. The method of claim 12, wherein the cavity liner or base is mixed with the glutaraldehyde to provide a semi-solid or solid bioactive glutaraldehyde carrier.

18. The method of claim 11, wherein the pulpitis is reversible or irreversible pulpitis.

19. The method of claim 11, wherein the tooth is a primary or permanent tooth.

20. A pharmaceutical composition for use to treat pulpitis and/or management of associated pain in a tooth, comprising a bioactive agent and a cavity liner or base.
